(19) United States
(12) Patent Application Publication
(10) Pub. No.: US 2006/0115528 A1
(43) Pub. Date: Jun. 1, 2006

(54) Rapidly disintegrating tablet

(76) Inventors: Vesna Skulj, Ljubljana (SI); Judita Sirca, Ljubljana (SI); Maja Jenko Osel, Kranj (SI)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/518,731

(22) PCT Filed: Jun. 20, 2003
(86) PCT No.: PCT/EP03/06528

§ 371 Date: Sep. 27, 2005

(30) Foreign Application Priority Data
Jun. 21, 2002 (SI) P-200200160

(51) Int. Cl. A61K 31/407; A61K 31/43; A61K 9/20

(52) U.S. Cl. 424/464; 514/192; 514/210.09

(57) ABSTRACT

The present invention relates to rapidly disintegrating tablets intended to be used as orodispersible tablets or dispersible tablets. They are ingested either by dispersing directly in the mouth or in water. The tablets include silicified microsrystalline cellulose. They are especially suitable for antibiotics. These tablets are also suitable for use in pediatric patients in the age above 3 years. For pediatric patients under 3 years the same tablets can be used as dispersible tablets. Rapidly disintegrating tablets which contain amoxicillin and clavulanic acid are also described.

The present invention belongs to the field of the pharmaceutical technology and it relates to rapidly disintegrating tablets intended to be used as orodispersible tablets or dispersible tablets which are dispersed in water prior to use.

Some patients, particularly pediatric and geriatric patients have difficulty swallowing or chewing solid dosage forms. Thus, there is a constant need for development of pharmaceutical formulations which rapidly disintegrate in the mouth of a patient and/or rapidly disperse in water.

Different techniques for preparation of rapidly disintegrating tablets are described in patent documents. The preparation of orodispersible tablets is generally technologically very demanding and expensive. Special, expensive manufacturing equipment is often needed. However, this formulation techniques appeared to be not applicable for the preparation of rapidly disintegrating tablets, containing a high amount of active ingredient. It is well known that for an effective treatment of diseases high doses of drugs and especially of antimicrobial compounds may have to be administered.

EP 910344 discloses fast-disintegrating and fast-dissolving compositions containing a high amount of drug. The active substance is incorporated in the granulate comprising water dispersible cellulose, which is microcrystalline cellulose and sodium carboxymethyl cellulose. The granulate is blended with a first and a second disintegrant, which is preferably selected from the group of superdisintegrants, and then compressed into tablets.

EP 80862 discloses water-dispersible composition of amoxicillin trihydrate and potassium clavulanate characterized in that it contains a non-hygroscopic water-soluble binder. Suitable binders are hydroxypropylmethyl cellulose, polyvinylpyrrolidone/polyvinyl acetate copolymer or hydroxypropylcellulose.

Dispersible tablets containing amoxicillin and clavulanic acid are described in WO 92/19227. These dispersible tablets are prepared as follows: The active substances are first roller-compacted together with a part of the excipients. The obtained granulate is then mixed with the remaining part of the excipients and the resulting mixture is compressed into tablets. Disintegrants used are the superdisintegrants such as, for example, cross-linked N-vinyl-2-pyrrolidone, croscarmellose sodium and/or sodium starch glycollate. In the description there is no data if the tablets are suitable to be used as orodispersible tablets.

WO 91/15197 discloses an effervescent formulation comprising amoxicillin trihydrat, potassium clavulanate and effervescent couple.

The patent applications WO 96/21429, WO 97/17947 and WO 99/15155 describe the conventional pharmaceutical compositions which comprise silicified microcrystalline cellulose. These tablets also include a superdisintegrant, e.g., croscarmellose sodium.

Thus it is the object of the present invention to overcome the problems encountered in the prior art and to provide improved rapidly disintegrating tablets.

### DESCRIPTION OF THE INVENTION

The above object is preferably achieved by a rapidly disintegrating tablet as specified in the claims. The present invention thus relates to rapidly disintegrating tablets suitable to be used as orodispersible tablets which are disintegrated in the oral cavity, or (and) as dispersible tablets which are dispersed in water prior to ingestion. The tablets may be taken directly in the mouth or used dispersed in a glass of water.

The novel pharmaceutical formulation is patient-friendly since it enables to choose between the two modes of administration. It is particularly suitable for children and elderly individuals and for those who have difficulty in swallowing. An advantage of the pharmaceutical composition is that it may be used in circumstances when drinking water is not available.

The manufacturing process of the tablets is very simple, the addition of superdisintegrants is not required. This technology enables preparation of the orodispersible tablets with high dosage of the active substance.

The pharmaceutical formulation of the invention is especially appropriate for antibiotics which are usually administered in high doses. As the pharmaceutical formulation which is disintegrated in the mouth it is also suitable for use in pediatric patients in the age above 3 years, and as the pharmaceutical formulation which is dispersed in water it may be also used in pediatric patients in the age under 3 years. For pediatric use the tablets of the invention (one or more tablets) of different strengths may be administered to the child so that the dose is adjusted according to the child's body weight (and age), severity of the infection and the causative microorganism which is determined empirically or in the laboratory.

The present invention provides rapidly disintegrating tablets which contain at least one active substance, silicified microcrystalline cellulose and optionally conventional excipients such as lubricants, desiccants, sweetening agents, flavours and colouring agents.

Advantageously the active substance is a drug which has to be administered in high doses. The active substance may be selected from the group of antibiotics comprising beta-lactam antibiotics from the groups of cephalosporins and penicillins (such as aminopenicillins, for example, amoxicillin and a combination of amoxicillin and clavulanic acid), macrolides, quinolones, aminoglycosides, tetracyclines and others. Preferable antibiotic is amoxicillin, alone or in a combination with clavulanic acid.

The dose of the active substance may vary depending on an individual active substance. The tablets may preferably contain up to 1500 mg of the active substance. The proportion of the active substances in the tablet is advantageously from 5 to 70% by weight of the tablet.

Silicified microcrystalline cellulose may be any commercially available form of this ingredient, for example, Prosolv SMCC, described in WO 96/21429 and manufactured by Penwest Company. There are different grades of silicified microcrystalline cellulose available. The amount of silicified microcrystalline cellulose in the tablets of the invention is about 30 to about 95% by weight of the tablets. A ratio of the active substance and silicified microcrystalline cellulose may preferably be in the range 0.5:1 to 2.5:1.

A lubricant is preferably selected from the group of hydrophobic lubricants such as hydrogenated fatty oils, magnesium stearate, and stearic acid. Especially suitable lubricant is selected from hydrogenated vegetable oils. Preferable lubricant is hydrogenated castor oil Cutina HR, Henkel. Sweetening agents optionally used in the tablets may be artificial sweetening agents such as, for example aspartame, saccharin sodium, acesulfame potassium or also natural sugars. Flavouring agents may preferably be selected from conventional flavours such as natural flavouring agents, nature-identical flavouring agents, and artificial flavouring agents of different tastes.

The tablets of the present invention may further include organic acids, for example, citric acid, desiccants, antiadhesives such as, for example, talc, glidants such as, for example, colloidal silicon dioxide, Aerosil 200.

If necessary, a particularly unpleasant taste of the active substances may be previously masked.

The process for preparation of the tablets of this invention is very simple. The active substance and excipients are blended; the mixture is homogenized, sieved and directly formed into tablets, preferably by compressing. Previous dry or wet granulation is not needed.

The tablets of the invention correspond to all pharmacopoeial standards for tablets, orodispersible tablets and dispersible tablets. The tablets are of the pleasant taste and rapidly disintegrate in the mouth or disperse in water. The physical characteristics of the tablets are suitable for packaging on a conventional packaging line which with rapidly disintegrating tablets manufactured by other technologies is not conventional.

The tablets of this invention may optionally be coated with a sufficiently thin and water soluble coating layer which does no influence on the ability of the tablet to disintegrate rapidly in the mouth. Suitable coating materials include disaccharides such as sucrose, polysaccharides such as maltodextrins and pectin, and cellulose derivatives such as hydroxypropyl cellulose and hydroxypropylmethyl cellulose.

The object of the present invention is also preferably achieved by rapidly disintegrating tablets which contain a combination of amoxicillin and clavulanic acid. The combination of the antibiotic amoxicillin and clavulanic acid, an inhibitor of beta-lactamase, is the well recognized and widely used medicament for treating bacterial infections in adult and pediatric patients. It is available in several dosage forms such as, for example, conventional tablets, chewable tablets, sachets, powder for preparation of oral suspension, dispersible tablets and controlled-release tablets.

The use of silicified microcrystalline cellulose has enabled manufacture of high dosage amoxicillin/clavulanic acid orodispersible and dispersible tablets with simple composition and technology.

Amoxicillin may be in the form of trihydrate or as crystalline sodium amoxicillin, clavulanic acid may be in the form of a salt, preferably potassium clavulanate. The ratio of amoxicillin and clavulanic acid is preferably from 2:1 to 30:1, especially suitable are the ratios 4:1, 7:1, 8:1, 12:1, 14:1 and 16:1.

The tablets may preferably contain from 250 to 1500 mg of amoxicillin and the appropriate amount of clavulanic acid. They may be prepared, for example, as tablets 250/125, 500/125, 500/62.5, 875/125, 1000/125, 1000/62.5, 400/57, 200/28.5, 250/62.5, 125/31.3. The weights being expressed as free parent acids amoxicillin and clavulanic acid.

The proportion of silicified microcrystalline cellulose in the tablet may preferably be from 30% to 90% by weight of the tablet. Preferably SMCC 90 is used.

An optional lubricant may be any lubricant from the group of hydrophobic lubricants. Especially suitable is hydrogenated castor oil Cutina HR, Henkel.

Special masking of the taste of active substances is not necessary. Conventional sweetening and flavouring agents may be added. The most convenient sweetening agents are aspartame and saccharin sodium. As the flavouring agent a combination of two flavours such as tropical blend and orange is favourable.

The tablets may also contain other excipients such as desiccants, glidants such as colloidal silicon dioxide, colouring agents, as occasion demands.

Generally the tablets which contain amoxicillin and clavulanic acid cannot be prepared by direct compression method. Usually one or both active substances and a part of excipients are pre-granulated by dry granulation process such as slugging or roller compaction. The granulate may then be mixed with the remaining part of the excipients and then the mixture is compressed into tablets. The tablets of the present invention may preferably be prepared according to the simple process by direct compression into tablets. Previous granulation is not necessary. All ingredients are blended, homogenized, sieved and directly formed, preferably compressed into tablets. As potassium clavulanate is highly moisture sensitive, previously dried ingredients should be used. The manufacturing of the tablets of the invention is preferably carried out under conditions of relative humidity not exceeding 25% RH, more suitably less than 20% RH.

Rapidly disintegrated tablets containing amoxicillin and clavulanic acid are also suitable for use in pediatric patients. Since they are of the pleasant taste and are simply ingested (dispersed directly in the mouth), they are suitable for children above 3 years old, and as the pharmaceutical formulation which is dispersed in water it may be also used in pediatric patients in the age under 3 years. The dosage should be adjusted according to child's body weight (and age), and severity of the infection and the causative microorganism determined empirically or in the laboratory.

The technology of preparation of the tablets of the invention provides formulating the tablets containing different unit doses of the active substance thus enabling the use of an appropriate dosage by administering the tablets of different strengths aimed at attaining the optimal dosage regarding the child's body weight (and age), severity of the infection and causative microorganism.

The tablets may replace the existing pediatric suspension formulations comprising amoxicillin and clavulanic acid.

For example, tablets containing 400 mg of amoxicillin and 57 mg of clavulanic acid may be prepared. One such tablet may replace the 5 ml of the existing pediatric suspension 400/57 for twice daily administration. Likewise, the tablet containing 200 mg of amoxicillin and 28.5 mg of clavulanic acid can replace the 5 ml of the existing pediatric suspension 200/28.5. One tablet containing 250 mg of amoxicillin and 62.5 mg of clavulanic acid can replace the 5 ml of existing pediatric suspension 250/62.5 per 5 ml. Likewise, the tablet containing 125 mg of amoxicillin and 31.25 mg of clavulanic acid can replace the 5 ml of the existing pediatric suspension 125/31.25 per 5 ml. The dosage in pediatric population with these tablets can be adjusted by taking a half of a tablet or a quarter of a tablet like in the case of suspensions where adjustments are possible by taking appropriate volumes of appropriate suspensions. The total daily dosage depends on the weight (and age) of a child, on the suspected microorganism causing the infection and on the severity of the infection.

The present invention is illustrated but in no way limited by the following examples:

### EXAMPLE 1

Composition of one tablet:

| INGREDIENTS | | |
|---|---|---|
| Amoxicillin (in the form of trihydrate) | 875 | mg |
| Clavulanic acid (in the form of potassium | 125 | mg |
| clavulanate) | | |
| Aspartame | 9 | mg |
| Flavour | 36 | mg |
| Aerosil 200 | 18 | mg |
| Cutina HR | 36 | mg |
| Talc | 18 | mg |
| Prosolv SMCC 90 | to 1932 | mg |

The method of manufacture:

All ingredients are blended, homogenized, sieved and compressed directly into tablets.

### EXAMPLE 2

Composition of one tablet:

| INGREDIENTS | | |
|---|---|---|
| Amoxicillin (in the form of trihydrate) | 500 | mg |
| Clavulanic acid (in the form of potassium | 125 | mg |
| clavulanate) | | |
| Aspartame | 6.5 | mg |
| Flavour | 26 | mg |
| Aerosil 200 | 13 | mg |
| Cutina HR | 26 | mg |
| Talc | 13 | mg |
| Prosolv SMCC 90 | to 1300 | mg |

All ingredients are blended, homogenized, sieved and compressed directly into tablets.

### EXAMPLE 3

Composition of one tablet:

| INGREDIENTS | | |
|---|---|---|
| Amoxicillin (in the form of trihydrate) | 437.5 | mg |
| Clavulanic acid (in the form of potassium | 62.5 | mg |
| clavulanate) | | |
| Aspartame | 4.5 | mg |
| Flavour | 18 | mg |
| Aerosil 200 | 9 | mg |
| Cutina HR | 18 | mg |
| Talc | 9 | mg |
| Prosolv SMCC 90 | to 966 | mg |

All ingredients are blended, homogenized, sieved and compressed directly into tablets.

### EXAMPLE 4

Composition of one tablet:

| INGREDIENTS | | |
|---|---|---|
| Amoxicillin (in the form of trihydrate) | 250 | mg |
| Clavulanic acid (in the form of potassium | 62.5 | mg |
| clavulanate) | | |
| Aspartame | 3.25 | mg |
| Flavour | 13 | mg |
| Aerosil 200 | 6.5 | mg |
| Cutina HR | 13 | mg |
| Talc | 6.5 | mg |
| Prosolv SMCC 90 | to 650 | mg |

All ingredients are blended, homogenized, sieved and compressed directly into tablets.

### EXAMPLE 5

Composition of one tablet:

| INGREDIENTS | | |
|---|---|---|
| Amoxicillin (in the form of trihydrate) | 1000 | mg |
| Clavulanic acid (in the form of potassium | 125 | mg |
| clavulanate) | | |
| Aspartame | 10.1 | mg |
| Flavour | 40.5 | mg |
| Aerosil 200 | 20.3 | mg |
| Cutina HR | 40.5 | mg |
| Talc | 20.3 | mg |
| Prosolv SMCC 90 | to 2174 | mg |

All ingredients are blended, homogenized, sieved and compressed directly into tablets.

### EXAMPLE 6

Composition of one tablet:

| INGREDIENTS | | |
|---|---|---|
| Amoxicillin (in the form of trihydrate) | 400 | mg |
| Clavulanic acid (in the form of potassium | 57 | mg |
| clavulanate) | | |
| Aspartame | 4.7 | mg |
| Flavour | 19 | mg |
| Aerosil 200 | 9.5 | mg |
| Cutina HR | 19 | mg |
| Talc | 9.5 | mg |
| Prosolv SMCC 90 | to 950 | mg |

All ingredients are blended, homogenized, sieved and compressed directly into tablets.

1. A rapidly disintegrating tablet comprising:
at least one active substance
silicified microcrystalline cellulose, and
optional excipients.

2. The rapidly disintegrating tablet according to claim 1, wherein the active substance is selected from the group of antibiotics.

3. The rapidly disintegrating tablet according to claim 1, wherein the active substance is amoxicillin in combination with clavulanic acid.

4. The rapidly disintegrating tablet according to claim 3, wherein the amoxicillin is in the form of amoxicillin 20 trihydrate.

5. The rapidly disintegrating tablet according to claim 3, wherein the clavulanic acid is in the form of potassium clavulanate.

6. The rapidly disintegrating tablet according to claim 3, wherein the ratio of amoxicillin to clavulanic acid is in the range of 2:1 to 30:1.

7. The rapidly disintegrating tablet according to claim 3, wherein the ratio of amoxicillin to clavulanic acid is 4:1.

8. The rapidly disintegrating tablet according to claim 3, wherein the ratio of amoxicillin to clavulanic acid is 7:1.

9. The rapidly disintegrating tablet according to claim 1, wherein the proportion of the active substance in the tablet is 5 to 70% by weight of the tablet.

10. The rapidly disintegrating tablet according to claim 1, wherein the ratio of the active substance and silicified microcrystalline cellulose is 0.5:1 to 2.5:1.

11. The rapidly disintegrating tablet according to claim 1 wherein the proportion of silicified microcrystalline cellulose is 30 to 95% by weight.

12. The rapidly disintegrating tablet according to claim 1, wherein hydrogenated castor oil is contained as a lubricant.

13. Use of the rapidly disintegrating tablet according to any of the preceding claims as an orodispersible tablet or as a dispersible tablet.

14. Use of the rapidly disintegrating tablet according to any of claims 1 to 12 in the manufacture of a medicament for the treatment of pediatric patients.

15. An orodispersible tablet comprising amoxicillin, clavulanic acid and silicified microcrystalline cellulose.

16. A dispersible tablet comprising amoxicillin, clavulanic acid and silicified microcrystalline cellulose.

17. A process for the manufacture of a rapidly disintegrating tablet according to claim 1 comprising the steps of:
blending the at least one active substance, silicified microcrystalline cellulose and optional excipients;
homogenizing the obtained mixture;
sieving the homogenized mixture; and
forming tablets therefrom.
